(19) United States
(12) Patent Application Publication
(10) Pub. No.: US 2010/0200670 A1
(43) Pub. Date: Aug. 12, 2010

(54) AIR FRESHENER FOR GARBAGE RECEPTACLES AND METHOD OF USE

(76) Inventor: Brandon Glenn Tipton, Springville, UT (US)

Correspondence Address: J. Todd Rushton, 1040 Bountiful Blvd., Bountiful, UT, 84010, US

(21) Appl. No.: 12/370,513

(22) Filed: Feb. 12, 2009

(51) Int. Cl. B65F 7/00; A61L 9/12

(52) U.S. Cl. 239/6; 239/57; 239/58; 239/60; 220/87.1

(57) ABSTRACT

A self-emptying air freshener device for use in garbage receptacles. The air freshener device is attached to the inside upper rim of a garbage receptacle and an air freshener element is placed inside. The air freshener element distributes the desired fragrance into the garbage receptacle. When the garbage receptacle is inverted to dump the contents into a garbage truck or larger bin, the lid of the air freshener device opens allowing the spent air freshener element to discharge. The user will place a new air freshener element into the air freshener device air after each time the garbage receptacle is emptied.

The present application relates to Provisional Application 61/028,903 filed, Feb. 15, 2008, and claims priority therefrom under 35 U.S.C. 119(e).

### BACKGROUND OF THE INVENTION

1. Field of the Invention

This invention relates to a garbage receptacle air freshener and method of use. More specifically, the invention relates to air fresheners apparatus having a vented body and self-closing lid that are attached to the inside of a garbage receptacle. An air freshener element is placed inside of the vented body and disseminates scent, deodorizing and freshening the garbage receptacle. When the garbage receptacle is full and discharged into a larger bin or garbage truck, the garbage receptacle is inverted to discharge the garbage, while inverted, the self-closing lid of the air freshener will open and the used air freshener element will be discharged and disposed of with the garbage. The user will then place a new air freshener element into the vented body and thus maintain a fresh smelling garbage receptacle.

2. Description of the Prior Art

Disposal of waste generated by everyday life has been an age-old problem. Early man would simply dispose of waste near his dwelling; the waste would accumulate and be subject to scavengers, vermin and insects. If the area became overly contaminated or the waste became odoriferous, the dweller would simply move to a new location until the waste naturally decomposed. Other early dwellers would use a river system or the rising and falling ocean tide to carry waste away from their home.

In the last century the amount and the nature of waste generated has changed dramatically. Each person on the earth generates hundreds, or many cases, thousands of pounds of garbage annually. Much of the garbage is toxic, and may take decades or even millennia to decompose. Society has adapted to handle the massive amounts of garbage. Most of it is deposited in sanitary landfills, some is incinerated, a portion is used as fuel for electrical power generation, and some of it is recycled. However, whatever the end result of the garbage is, all garbage has to be handled from the point of origin until it reaches a place for final disposal or rehandling.

Most garbage is first disposed in small open containers within a home or structure and then moved to larger containers that are placed outside or in a garage. The larger container is used to store the garbage until a scheduled garbage collection service, provided by a municipality or a private contractor. During collection the large container is emptied into a garbage truck and transported away. Garbage pick up is normally scheduled every seven days but may extend to 14 days or longer.

Most garbage will have some odor. Some garbage, such as soiled diapers or animal waste, may be quite repugnant when first placed in the garbage receptacle. Almost all garbage will become odorous when it is stored and is allowed to begin the natural decomposition process. Decomposition is accelerated by environmental conditions such as, ultra-violet light, high humidity or heat. Almost everyone has experienced the unpleasant smell of a full, black plastic, garbage can on hot summer day.

Some have tried to remedy the garbage odor problem by using an air freshener device in the garbage receptacle such as those shown in U.S. Pat. No. 3,840,145 to Almanzana, U.S. Pat. No. 4,279,373 to Montealegre, or U.S. Pat. No. 5,988,520 to Bitner. After the air freshener element in these devices is spent, the user is required to either, inconveniently open the air freshener holder and replace the air freshener element, or to replace the entire device.

What is needed, is an effective air freshener that will eliminate odors and is convenient for the user.

### SUMMARY OF THE INVENTION

The present invention and method of use helps remedy the aforementioned problems of odorous garbage receptacles and inconvenient air fresheners. The invention comprises a vented body designed to accommodate an air freshener element. The vented body is secured to the upper edge on the inside of the garbage receptacle using a bracket attached or formed integral to the vented body. It is also contemplated that the vented air freshener body is attached with screws, bolts, adhesive, hook and loop fastener, or is formed integral with the garbage receptacle. A self-closing lid covers the vented body to retain the air freshener element until the garbage receptacle is fully inverted and to prevent garbage from being deposited into the vented body. The self-closing lid may be biased toward the closed position by gravity, by use of a spring or with a counterweight. The counterweight can extend below the vented body, be contained within the vented body or the counterweight can be provided by the air freshener element. Possible cross-sections of the vented body are cylindrical, semi-cylindrical, oval, square or rectangular. The profile of the vented body includes a rounded or angled bottom surface to allow garbage flow past the vented body without catching or snagging. The venting orifices in the body can be a fixed aperture or adjustable by means of a movable overlaying band with matching orifices. If an overlaying band is used the vent aperture size can be adjusted by the user by simply moving the overlaying band to offset the amount of alignment between the matching orifice sets. The air freshener element is designed with sufficient mass and in a shape that is conducive to discharging out of the vented body when the garbage receptacle is inverted. Shapes considered for the air freshener element, include a sphere, egg, disc, cube, rectangular block, prism, rod, cone, pyramid, dodecahedron and other three-dimensional shapes.

The air freshener is used by attaching the vented body to the inside upper rim of the garbage receptacle. This may be accomplished with a hanger bracket or the vented body may be snapped, screwed or bolted through holes drilled through the side of the garbage receptacle. The inventor also contemplates a receiver formed into the side of the garbage receptacle, in combination with a mating flange on the vented body, for direct attachment of the vented body to the garbage receptacle or forming the vented body integral with the garbage receptacle. To charge the air freshener, the user lifts the air freshener lid and places an air freshener element into the vented body. When the lid is released, it will close automatically. If the vented body is adjustable, the user can rotate the overlaying band and control the size of the vent orifices. This can be adjusted at anytime depending upon the amount of fragrance desired by the user. The fragrance will emanate from the vented body and will be strongest when first placed into the vented body and will dissipate with time as the essential oils or fragrance in the element vaporizes. The amount of fragrance will vary with temperature, less fragrance in cold temperatures and more fragrance when the weather is hot. When the garbage receptacle is either mechanically or manually emptied into a garbage truck or larger bin, the garbage receptacle and air freshener unit will be inverted, causing the self-closing lid to open and the spent air freshener element to discharge with the garbage. The user will again open the self-closing lid and place a new air freshener element into the vented body. Air freshener elements will be separately wrapped to preserve the fragrance and will be opened when the user desires to place a new element into the vented body.

### BRIEF DESCRIPTION OF DRAWINGS

FIG. 1 is a side view of the self-emptying air freshener device.

FIG. 2 is a top view of the self-emptying air freshener device.

FIG. 3 cross-sections of possible air freshener elements.

### DETAILED DESCRIPTION OF THE INVENTION

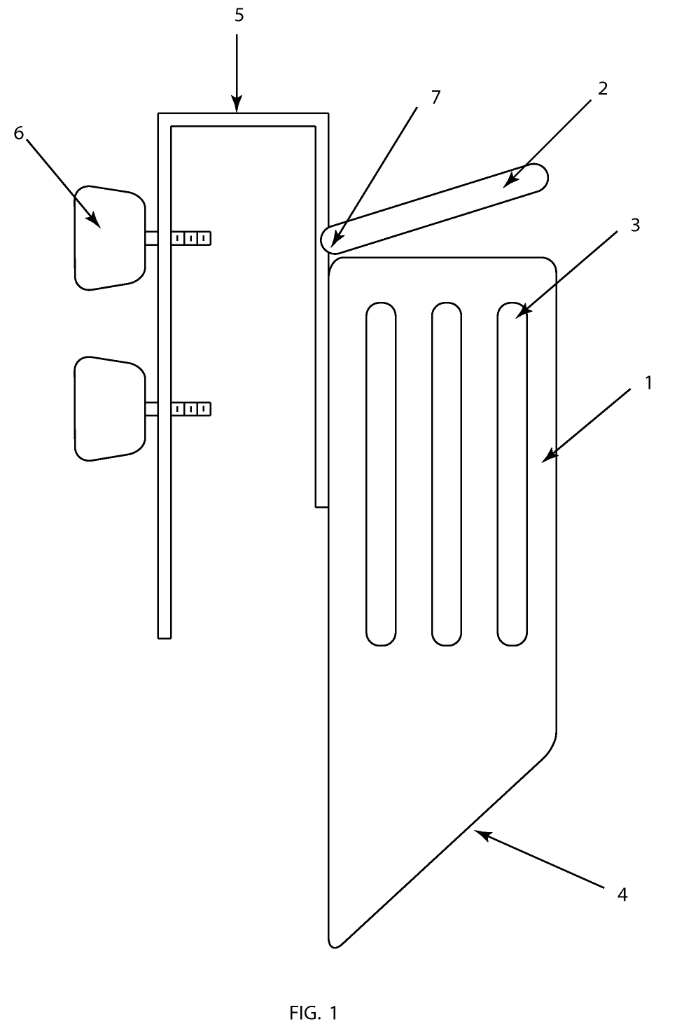

A self-emptying air freshener device for use in garbage receptacles as shown in FIG. 1, the device includes a vented body I in which an air freshener element is placed, self-closing lid 2 is raised to allow access to the vented body 1. Once an air freshener element is installed, fragrance will emanate through the vent openings 3, vent openings 3 can have a fixed aperture or may be adjustable by use of an overlapping band having vent openings that match the vented body 1, the overlapping band can be turned in order to adjusted the amount of interference between the matching vent openings. The change in interference allows the user to control the amount of fragrance discharged. It is contemplated that vented body I includes a slanted or rounded bottom surface 4, allowing garbage to slide past the air freshener device without catching or snagging. Self-closing cover 2 includes a hinge point 7 and is biased in the closed position by gravity, a spring or may include a counterweight as commonly known in the art. The air freshener device is attached to the garbage receptacle with bracket 5, which overlaps the rim of the garbage receptacle and is secured in place with thumbscrews 6. Additional methods of attachment include screws, bolts, adhesive, hook and loop fastener, integrated foot and mating slot, or the device may be formed integral with the garbage receptacle.

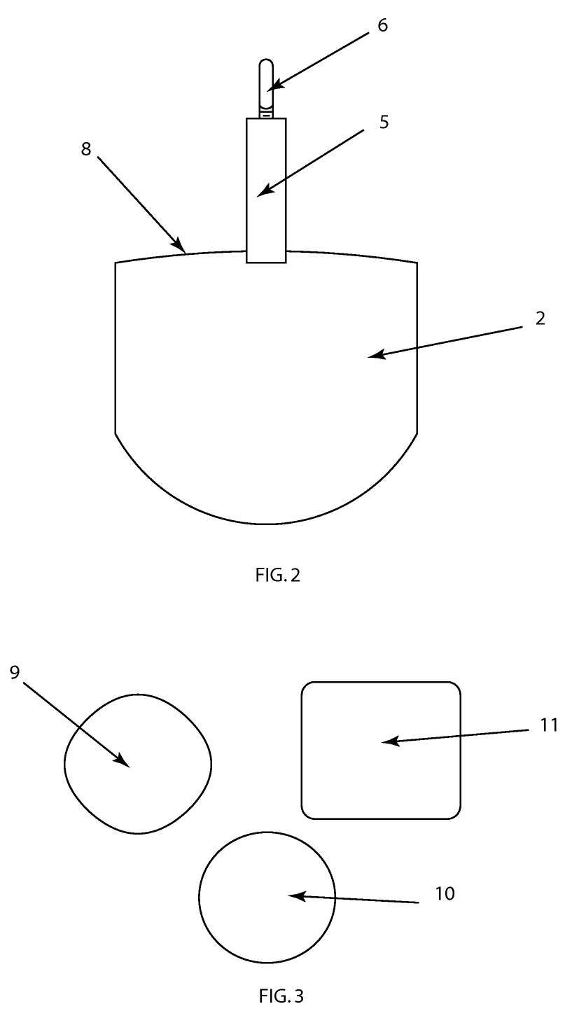

FIG. 2 is a top view of the air freshener device and shows self-closing lid 2 and bracket 5 with thumbscrews 6. The back surface 8 of the air freshener device can be flat, rounded or contoured as shown, to mate more precisely with a specific garbage receptacle.

FIG. 3 includes cross-sections of air freshener elements, which may be an irregular rounded section 9, circular section 10 or a rectangular section 11. It is contemplated that any shape that is conducive to rolling, sliding or generally discharging out the vented body, while the vented body is inverted, may be used.

1. An air freshener device comprising, a self-emptying vented body, an attachment mechanism, a replaceable air freshener element and a self-closing lid.

2. The air freshener device of claim 1 wherein, the vented body includes sides, an open top and a closed bottom.

3. The air freshener device of claim 2 wherein, the closed bottom is one of slanted, tapered, contoured and rounded.

4. The air freshener device of claim 2 wherein, the vented body includes fixed aperture vent orifices.

5. The air freshener device of claim 2 wherein, the vented body includes adjustable aperture vent orifices.

6. The air freshener device of claim 1 wherein, the self-closing lid is biased closed by gravity when the air freshener device is in an upright position.

7. The air freshener device of claim 1 wherein, the self-closing lid is biased closed by a spring.

8. The air freshener device of claim 1 wherein, the self-closing lid is biased closed by a counterweight when the air freshener device is in an upright position.

9. The air freshener device of claim 1 wherein, the attachment mechanism is one of a bracket, hook and loop fastener, bolt and nut, screws, snaps, adhesive, and integrated foot and mating slot engagement.

10. The air freshener device of claim 1 wherein, the replaceable air freshener element forms a sphere, egg, disc, cube, rectangular block, prism, rod, cone, pyramid and dodecahedron.

11. A method of using a garbage can air freshener including, attaching an air freshener assembly, having a self-emptying vented body and a self-closing lid, to the inside upper rim of a garbage receptacle while the garbage receptacle is in an upright position, lifting the self-closing lid and placing the air freshener element inside of the vented body, inverting the garbage receptacle allowing the self-closing lid to open and the air freshener element to discharge out of the vented body, returning the garbage receptacle to the upright position, lifting the self-closing lid and placing a second air freshener element into the vented body.
